(19) United States
(12) Patent Application Publication
(10) Pub. No.: US 2026/0108504 A1
(43) Pub. Date: Apr. 23, 2026

(54) ENTERIC COATED TABLET WITH ACECLOFENAC AND PARACETAMOL TABLET AND PROCESS OF PREPARATION THEREOF

(71) Applicant: ATOZ Pharmaceuticals Pvt., Ltd., Chennai (IN)

(72) Inventors: Olaganathan Arumugam, TN (IN); Arjun Arumugam, Chennai (IN); Arun Balakrishnan Elango, Chennai (IN)

(21) Appl. No.: 19/009,980

(22) Filed: Jan. 4, 2025

(30) Foreign Application Priority Data
Oct. 22, 2024 (IN) 202441080311

(51) Int. Cl. A61K 31/4439; A61K 9/20; A61K 9/28; A61K 31/167; A61K 31/216

(52) U.S. Cl. CPC A61K 31/4439; A61K 9/2013; A61K 9/2027; A61K 9/2054; A61K 9/2059; A61K 9/2813; A61K 9/282; A61K 9/284; A61K 9/286; A61K 9/2893; A61K 31/167; A61K 31/216

(57) ABSTRACT

Embodiments herein provide a pharmaceutical formulation comprising an anti-inflammatory antipyretic agent with reduced side effects, addressing gastric problems. An enteric-coated tablet of the present invention comprises esomeprazole, aceclofenac, and paracetamol, along with maize starch, microcrystalline cellulose, povidone K30, colloidal anhydrous silica, and magnesium stearate.

### FIELD OF INVENTION

The present disclosure relates to pharma, and more particularly to an enteric coated tablet with aceclofenac and paracetamol tablet and process of preparation thereof. The present application is based on, and claims priority from Indian Application No. 202441080311 filed on 22 Oct. 2024, the disclosure of which is hereby incorporated by reference.

### BACKGROUND

An enteric-coated tablet containing Aceclofenac and Paracetamol is designed to provide effective pain relief with reduced gastrointestinal side effects. The process involves careful selection of excipients, granulation, tablet compression, and enteric coating to ensure the final product meets pharmaceutical standards for efficacy, safety, and patient compliance. This formulation approach exemplifies modern pharmaceutical technology aimed at enhancing drug delivery and patient outcomes.

An enteric-coated tablet containing Aceclofenac and Paracetamol combines two active pharmaceutical ingredients (APIs) with specific characteristics and therapeutic purposes. Aceclofenac is a non-steroidal anti-inflammatory drug (NSAID) with analgesic and anti-inflammatory properties. It is primarily used to relieve pain and inflammation in conditions such as osteoarthritis, rheumatoid arthritis, and ankylosing spondylitis. However, Aceclofenac can cause gastric irritation and ulcers due to its action on prostaglandin synthesis. Paracetamol is an analgesic and antipyretic agent that works centrally to reduce pain and fever. It is commonly used to manage mild to moderate pain and fever. Paracetamol is generally safer for the gastrointestinal tract compared to NSAIDs like Aceclofenac.

Aceclofenac can cause irritation and damage to the stomach lining due to inhibition of prostaglandin synthesis. Enteric coating prevents the release of the drug in the stomach, directing it to the small intestine where pH conditions are less acidic, reducing the risk of irritation. Enteric-coated tablets are easier to swallow and can improve patient adherence to treatment due to reduced gastrointestinal side effects.

Aceclofenac, Paracetamol, and excipients are mixed thoroughly by dry mixing. Further, the composition can be made by granulating the mixture using a solvent to form granules. This step improves flow properties and uniformity. The wet granules are dried to remove the solvent. The dried granules are compressed into tablets using a tablet press. Lubricants are often added to prevent sticking.

Gaikwad and Kshirsagar, Beni-Suef University Journal of Basic and Applied Sciences (2020) 9:1, disclose that film and sugar coatings have a number of disadvantages, the most important one being the utilization of aqueous or organic solvent that leads to toxicity. To overcome this problem, in the year 1896, Noyes first introduced the compression coating or Tablet in Tablet technique. In the development of Tablet in Tablet dosage form, substantial attention among researchers and various research reports and patents inputs can be found in the literature. Also, we focused on the recent advancements in techniques like one-step dry-coating (OSDrC) for manufacturing Tablet in Tablet dosage form.

U.S. Pat. No. 8,747,920B2 discloses pharmaceutical compositions comprising a non-steroidal anti-inflammatory drug, antipyretic-analgesic drug, and proton pump inhibitor. The document relates to pharmaceutical compositions in the form of a fixed combination comprising a non-steroidal anti-inflammatory drug or its single enantiomers or salts thereof, antipyretic-analgesic drug, and proton pump inhibitor or its single enantiomers or salts thereof. The invention also relates to processes for the preparation of such compositions.

US2005/0249806A1 discloses pharmaceutical compositions comprising a proton pump inhibitor, one or more buffering agents, and a nonsteroidal anti-inflammatory drug. Methods are described for treating gastric acid-related disorders and treating inflammatory disorders using pharmaceutical compositions comprising a proton pump inhibitor, a buffering agent, and a nonsteroidal anti-inflammatory drug.

Therefore, there arises a need to develop a better composition with reduced side effects. Given the above, there arises a need to develop a formulating tablet in tablet dosage form with reduced side effects and overcome the gastric problem. The present invention provides a better solution for the above.

### OBJECT OF INVENTION

The principal object of the embodiments herein is to provide an enteric-coated tablet with aceclofenac and paracetamol and a process of preparation thereof.

### SUMMARY

In an aspect, the embodiments herein provide that Aceclofenac and Paracetamol work by lowering the level of those chemical substances in the body which cause pain and inflammation. Esomeprazole helps in preventing the digestive problems that may occur due to Aceclofenac. The orally administered formulation comprises a therapeutically effective amount of a pharmaceutical composition in the form of a tablet in tablet.

The present invention provides an enteric-coated tablet comprising a core comprising esomeprazole, maize starch, microcrystalline cellulose, and povidone K30, and a shell over the core comprising aceclofenac and paracetamol, maize starch, and povidone K30.

In an embodiment, the present invention provides that the tablet comprises 10-65 percentage weight of Esomeprazole magnesium trihydrate based on the total weight of the core.

The enteric-coated tablet as claimed in claim 1, wherein the maize starch ranges in an amount of 2-35 percentage weight based on the total weight of the core.

In an embodiment, the present invention provides that the microcrystalline cellulose ranges from 30-50 percentage weight based on the total weight of the core.

In an embodiment, the present invention provides that the Povidone K30 ranges from 0-5 percentage weight based on the total weight of the core.

In an embodiment, the present invention provides that the colloidal anhydrous silica ranges from 0-3 percentage weight based on the total weight of the core.

In an embodiment, the present invention provides that the magnesium stearate is 0-3 percentage weight based on the total weight of the core.

In an embodiment, the present invention provides that the Aceclofenac ranges from 5-30 percentage weight based on the total weight of the tablet in tablet.

In an embodiment, the present invention provides that the Paracetamol ranges from 40-80 percentage weight based on the total weight of the tablet in tablet.

In an embodiment, the present invention provides that the pharmaceutical composition of Esomeprazole tablet coated with gastro-resistant coating comprises 3-8 percentage weight of Methacrylic Acid—Ethyl Acrylate Copolymer (1:1) Dispersion 30 Percent.

In an embodiment, the present invention provides that the pharmaceutical composition of tablet in tablet is coated with 15-5 percent weight of Hydroxypropyl methylcellulose (HPMC) with iron oxide yellow.

In another aspect, the present invention provides a process for preparing tablet in tablet dosage form. Said process comprises obtaining a tablet dosage form of Esomeprazole and coating with gastro-resistance by blending and compressing Esomeprazole magnesium trihydrate, maize starch, microcrystalline cellulose, and Povidone K30, blending Aceclofenac, Paracetamol, maize starch, and Povidone K30 to obtain a uniform mixture, compressing the tablet obtained in step (i) and the blend obtained in step (ii) to obtain Esomeprazole enteric-coated tablet in Aceclofenac and Paracetamol tablet.

In an embodiment, the present invention provides that the blending is performed for a period of 10-20 minutes at 10-20 rpm to obtain a uniform mixture.

In an embodiment, the present invention provides that the uniform mixture is lubricated with colloidal anhydrous silica and magnesium stearate for 5-15 minutes at 10-20 rpm.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the invention thereof, and the embodiments herein include all such modifications.

### BRIEF DESCRIPTION OF FIGURES

This invention is illustrated in the accompanying drawings, throughout which like reference letters indicate corresponding parts in the various figures. The embodiments herein will be better understood from the following description with reference to the drawings, in which:

FIG. 1 illustrates the process flowchart for Esomeprazole Enteric Coated Tablets.

FIG. 2 illustrates the process flowchart for Aceclofenac & Paracetamol Granules, and

FIG. 3 illustrates the process flowchart for Aceclofenac, Paracetamol & Esomeprazole (as Enteric Coated) Tablets.

It may be noted that to the extent possible, like reference numerals have been used to represent like elements in the drawing. Further, those of ordinary skill in the art will appreciate that elements in the drawing are illustrated for simplicity and may not have been necessarily drawn to scale. For example, the dimension of some of the elements in the drawing may be exaggerated relative to other elements to help to improve the understanding of aspects of the invention. Furthermore, the elements may have been represented in the drawing by existing symbols, and the drawings may show only those specific details that are pertinent to the understanding the embodiments of the invention so as not to obscure the drawing with details that will be readily apparent to those of ordinary skill in the art having benefit of the description herein.

### DETAILED DESCRIPTION OF INVENTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein, refers to a non-exclusive or, unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

The accompanying drawings are used to help easily understand various technical features and it should be understood that the embodiments presented herein are not limited by the accompanying drawings. As such, the present disclosure should be construed to extend to any alterations, equivalents and substitutes in addition to those which are particularly set out in the accompanying drawings. Although the terms first, second, etc. may be used herein to describe various elements, these elements should not be limited by these terms. These terms are generally only used to distinguish one element from another.

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. Also, the various embodiments described herein are not necessarily mutually exclusive, as some embodiments can be combined with one or more other embodiments to form new embodiments. The term “or” as used herein refers to a non-exclusive “or” unless otherwise indicated. The examples used herein are intended merely to facilitate an understanding of ways in which the embodiments herein can be practiced and to further enable those skilled in the art to practice the embodiments herein. Accordingly, the examples should not be construed as limiting the scope of the embodiments herein.

In the realm of pharmaceutical formulations, the combination of active ingredients to enhance therapeutic efficacy and minimize adverse effects is a critical area of innovation. The embodiment herein addresses a common challenge associated with the administration of nonsteroidal anti-inflammatory drugs (NSAIDs), such as Aceclofenac, which are known to potentially cause gastric irritation or ulcers. By integrating esomeprazole, a proton pump inhibitor, into a tablet dosage form alongside Aceclofenac and paracetamol, the formulation aims to mitigate the gastrointestinal side effects often induced by NSAID therapy. This innovative approach not only enhances patient compliance by simplifying medication regimens but also provides a more holistic treatment option by addressing both pain and potential gastric discomfort simultaneously.

The formulation process involves the enteric coating of esomeprazole, which ensures its stability and targeted release in the intestine rather than the stomach. This is crucial for maintaining the integrity and efficacy of esomeprazole, as it is susceptible to degradation in the acidic environment of the stomach. The enteric coating thus allows for the delayed release of esomeprazole, aligning with its therapeutic action to reduce gastric acid secretion and protect the gastric mucosa. Meanwhile, Aceclofenac and paracetamol serve their roles as anti-inflammatory and analgesic agents, respectively, providing relief from pain and inflammation. The strategic combination of these components within a single tablet not only streamlines the treatment process for patients but also exemplifies the potential of pharmaceutical innovation to enhance therapeutic outcomes and patient quality of life.

Furthermore, the development of such a combination tablet requires meticulous attention to the pharmacokinetics and pharmacodynamics of each component to ensure optimal efficacy and safety. The formulation must consider the absorption rates, onset of action, and duration of effect for each active ingredient to achieve a balanced therapeutic profile. This involves sophisticated techniques in pharmaceutical technology, such as granulation, compression, and coating, to produce a stable and effective dosage form. The embodiment herein underscores the importance of interdisciplinary collaboration in drug development, drawing upon expertise in chemistry, pharmacology, and materials science to create a product that meets the complex needs of patients. Through such innovations, the pharmaceutical industry continues to advance towards more personalized and effective treatment options, ultimately improving patient outcomes and quality of care.

The present invention provides an enteric-coated tablet comprising esomeprazole, aceclofenac, and paracetamol, maize starch, microcrystalline cellulose, povidone K30, colloidal anhydrous silica, and magnesium stearate. The tablet comprises 10-65 percentage weight of esomeprazole magnesium trihydrate based on the total weight of the core tablet.

The maize starch ranges in an amount of 2-35 percentage weight based on the total weight of the whole tablet. The microcrystalline cellulose ranges from 30-50 percentage weight based on the total weight of the core tablet. The povidone K30 ranges from 0-5 percentage weight based on the total weight of the whole tablet. The colloidal anhydrous silica ranges from 0-3 percentage weight based on the total weight of the whole tablet. The magnesium stearate ranges from 0-3 percentage weight based on the total weight of the whole tablet. The aceclofenac ranges from 5-30 percentage weight based on the total weight of the whole tablet. The paracetamol ranges from 40-80 percentage weight based on the total weight of the whole tablet. The pharmaceutical composition of the esomeprazole tablet coated with a gastro-resistant coating comprises 3-8 percentage weight of Methacrylic Acid—Ethyl Acrylate Copolymer (1:1) Dispersion 30 Percent.

The pharmaceutical composition described in the present invention involves a sophisticated tablet-in-tablet dosage form, which is meticulously engineered to deliver a combination of active pharmaceutical ingredients (APIs) with distinct release profiles. The outer layer of the tablet is coated with 15-5 percent by weight of Hydroxypropyl methylcellulose (HPMC) combined with iron oxide yellow. This coating not only provides a protective barrier to the inner core but also contributes to the aesthetic appeal of the tablet by imparting a distinct yellow color. HPMC is a well-known polymer used in pharmaceutical formulations for its film-forming properties, which aid in controlling the release rate of the APIs, ensuring that the active ingredients are delivered at the desired site of action within the gastrointestinal tract.

In one embodiment, the invention outlines a detailed process for preparing the tablet-in-tablet dosage form. Initially, Esomeprazole magnesium trihydrate is blended with excipients such as Maize starch, Microcrystalline cellulose, and Povidone K30. This blending process, conducted for 10 to 20 minutes at a speed of 10 to 20 rpm, ensures a uniform distribution of the active ingredient throughout the mixture. The uniform mixture is then lubricated with Colloidal anhydrous silica and magnesium stearate for an additional 5 to 15 minutes at the same rpm range. This lubrication step is crucial as it enhances the flow properties of the powder blend, reduces friction during compression, and prevents sticking to the tablet punches. The lubricated blend is then compressed into a tablet form, which subsequently undergoes a gastro-resistant coating process. This coating is essential for protecting Esomeprazole from the acidic environment of the stomach, allowing it to reach the intestine where it can be absorbed effectively.

The process further involves the preparation of an outer tablet layer comprising Aceclofenac and Paracetamol. These APIs are blended with Maize starch and Povidone K30 under similar conditions to achieve a uniform mixture. The mixture is then lubricated with Colloidal anhydrous silica and magnesium stearate, followed by compression to form the outer tablet. The final step involves combining the Esomeprazole enteric-coated tablet with the Aceclofenac and Paracetamol tablet to create the tablet-in-tablet dosage form. This innovative design allows for the simultaneous delivery of a proton pump inhibitor and analgesic agents, offering a comprehensive therapeutic approach for conditions requiring both gastric acid suppression and pain relief. The unique formulation not only enhances patient compliance by reducing the pill burden but also optimizes the pharmacokinetic profiles of the included drugs, ensuring maximum therapeutic efficacy.

### Example 1

Blending and compressing 30% of Esomeprazole magnesium trihydrate, 15-20% of maize starch, 30-50% of microcrystalline cellulose, and 1-5% of Povidone K30 for 15 minutes at 20 rpm to obtain a uniform mixture. Compressing and coating with a gastro-resistant coating to obtain a tablet dosage form of Esomeprazole enteric-coated. Blending 15% of Aceclofenac, 75% of Paracetamol, 2-15% of maize starch, and 1-5% Povidone K30 for 15 minutes at 20 rpm to obtain a uniform mixture.

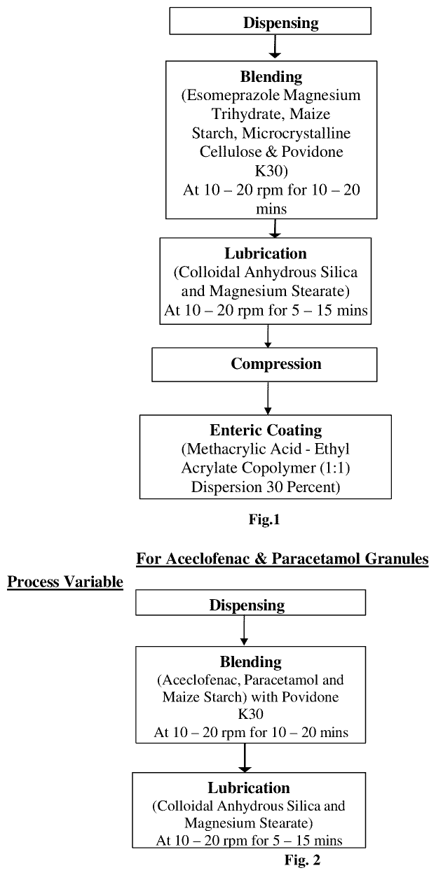

The process flowcharts depicted in FIGS. 1, 2, and 3 provide a comprehensive overview of the manufacturing processes for Esomeprazole Enteric Coated Tablets, Aceclofenac & Paracetamol Granules, and a combined formulation of Aceclofenac, Paracetamol, and Esomeprazole tablets, respectively. These flowcharts outline the precise steps and conditions necessary to achieve the desired pharmaceutical formulations, ensuring both efficacy and stability of the active ingredients.

In FIG. 1, the process begins with the careful dispensing and blending of Esomeprazole Magnesium Trihydrate, Maize Starch, Microcrystalline Cellulose, and Povidone K30. This initial blending step is crucial as it ensures uniform distribution of the active pharmaceutical ingredient (API) and excipients, which is essential for consistent drug release and bioavailability. The blending speed and duration, set at 10 to 20 rpm for 10 to 20 minutes, are optimized to achieve a homogenous mixture without causing degradation of sensitive components. Following this, Colloidal Anhydrous Silica and Magnesium Stearate are incorporated as lubricants. These agents facilitate the smooth compression of the blend by reducing friction between particles and the tablet press surfaces, thus preventing sticking and ensuring the integrity of the tablet shape. The final step involves compressing the blend to form tablets, which are then enteric-coated using a Methacrylic Acid—Ethyl Acrylate Copolymer dispersion. This enteric coating is vital as it protects the Esomeprazole from degradation in the acidic environment of the stomach, allowing it to reach the intestine where it can be absorbed effectively.

FIG. 2 details the preparation of Aceclofenac and Paracetamol granules. Similar to the process in FIG. 1, the active ingredients Aceclofenac and Paracetamol are blended with Maize Starch and Povidone K30. This mixture is blended at controlled speeds to ensure uniformity, which is critical for the therapeutic efficacy of the final product. The addition of Colloidal Anhydrous Silica and Magnesium Stearate as lubricants is a common practice in pharmaceutical manufacturing, as it enhances the flow properties of the granules and prevents issues during the subsequent compression process. The precise control of blending parameters ensures that the granules possess the desired mechanical properties and dissolution profiles.

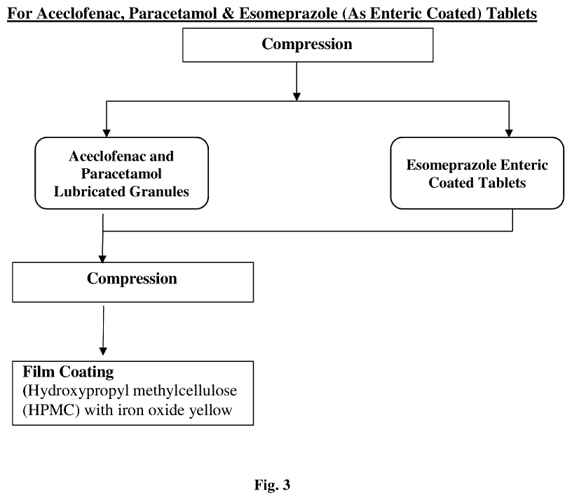

In FIG. 3, the process culminates with the compression of the lubricated granules of Aceclofenac and Paracetamol along with the enteric-coated Esomeprazole tablets to form a multi-ingredient tablet. This step is particularly challenging as it requires careful consideration of the physical and chemical compatibility of the different components. The final product is then film-coated with Hydroxypropyl Methylcellulose (HPMC) and iron oxide yellow, which not only provides an aesthetically pleasing appearance but also offers additional protection against environmental factors such as moisture and light. The film coating also aids in masking any unpleasant taste and facilitates easier swallowing for patients. The integration of these processes highlights the complexity and precision involved in pharmaceutical manufacturing, ensuring that the final product meets stringent quality standards and delivers the intended therapeutic benefits.

Compressing the tablet dosage form of Esomeprazole and the blend of Aceclofenac and Paracetamol to obtain Esomeprazole enteric-coated tablet in Aceclofenac and Paracetamol tablet

| | Composition 1 | Composition 2 | Composition 3 |
|---|---|---|---|
| Composition | (mg) | (mg) | (mg) |
| Esomeprazole; | 23.9 | 23.9 | 23.9 |
| Dry Granulation | | | |
| Maize starch | 18 | 12 | 28 |
| Microcrystalline | 35 | 40 | 24 |
| cellulose | | | |
| Granulation with | | | |
| binding agent | | | |
| Povidone K30 | 3 | 1 | 4 |
| IPA | Qs | Qs | Qs |
| Lubrication | | | |
| Colloidal | 1 | 2.4 | |
| anhydrous silica | | | |
| Magnesium | 1 | | 2.4 |
| stearate | | | |
| Gastro-resistance | | | |
| coating | | | |
| Methacrylic acid - | 5 | 3 | 7 |
| Ethyl acrylate | | | |
| copolymer (1:1) | | | |
| dispersion 30% | | | |
| Dichloromethane | Qs | Qs | Qs |
| Total weight | 87 | 82 | 89 |
| with coating | | | |
| Aceclofenac; | 100 | 100 | 100 |
| Paracetamol; | 500 | 500 | 500 |
| Dry Granulation | | | |
| Maize starch | 55 | 13 | 90 |
| Granulation with | | | |
| binding agent | | | |
| Povidone K30 | 15 | 13 | 33 |
| Water | Qs | Qs | Qs |
| Lubrication | | | |
| Colloidal | 5 | 5 | 15 |
| anhydrous silica | | | |
| Magnesium | 5 | 15 | 5 |
| stearate | | | |
| Coating | | | |
| Hydroxypropyl | 25 | 39 | 12 |
| methylcellulose | | | |
| (HPMC) with | | | |
| iron oxide yellow | | | |
| Water | Qs | Qs | Qs |
| Total weight of | 792 | 767 | 844 |
| Tablet in Tablet | | | |

The foregoing description of the specific embodiments will so fully reveal the general nature of the embodiments herein that others can, by applying current knowledge, readily modify and or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described herein.

We claim:

1. An enteric coated tablet comprising:
a. a core comprising:
i. esomeprazole;
ii. maize starch;
iii. microcrystalline cellulose; and
iv. povidone k30;
v. colloidal anhydrous silica;
vi. magnesium stearate
b. a shell over the core comprising:
i. aceclofenac and paracetamol;
ii. maize starch; and
iii. povidone k30;
iv. colloidal anhydrous silica;
v. magnesium stearate.

2. The enteric coated tablet as claimed in claim 1, wherein the tablet comprises 10-65 percentage weight of Esomeprazole magnesium trihydrate based on total weight of the core.

3. The enteric coated tablet as claimed in claim 1, wherein the maize starch ranges in an amount of 2-35 percentage weight based on total weight of the core.

4. The enteric coated tablet as claimed in claim 1, wherein the Microcrystalline cellulose ranges from 30-50 percentage weight based on total weight of the core.

5. The enteric coated tablet as claimed in claim 1, wherein the Povidone K30 ranges from 0-5 percentage weight based on total weight of the core.

6. The enteric coated tablet as claimed in claim 1, wherein the Colloidal anhydrous silica ranges from 0-3 percentage weight based on total weight of the core.

7. The enteric coated tablet as claimed in claim 1, wherein the Magnesium stearate 0-3 percentage weight of based on total weight of the core.

8. The enteric coated tablet as claimed in claim 1, wherein the Aceclofenac ranges from 5-30 percentage weight based on total weight of the tablet in tablet.

9. The enteric coated tablet as claimed in claim 1, wherein the Paracetamol ranges from 40-80 percentage weight based on total weight of the tablet in tablet.

10. The enteric coated tablet as claimed in claim 1, wherein pharmaceutical composition of Esomeprazole tablet coated with gastro-resistant coating comprises 3-8 percentage weight of Methacrylic Acid—Ethyl Acrylate Copolymer (1:1) Dispersion 30 Percent.

11. The enteric coated tablet as claimed in claim 1, wherein pharmaceutical composition of tablet in tablet is coated with 1.5-5 percent weight of Hydroxypropyl methylcellulose (HPMC) with iron oxide yellow.

12. A process for preparing tablet in tablet dosage form, said process comprises:
i. obtaining a tablet dosage form of Esomeprazole and coating with gastro-resistance by blending and compressing Esomeprazole magnesium trihydrate, Maize starch, Microcrystalline cellulose and Povidone K30;
ii. blending Aceclofenac, Paracetamol, Maize starch and Povidone K30 to obtain uniform mixture;
iii. compressing the tablet obtained in step (i) and the blend obtained in step (ii) to obtain Esomeprazole enteric coated tablet in Aceclofenac and Paracetamol tablet.

13. The process as claimed in claim 12, wherein the blending is performed for a period of 10-20 minutes at 10-20 rpm to obtain uniform mixture.

14. The process as claimed in claim 12, wherein the uniform mixture is lubricated with Colloidal anhydrous silica and magnesium stearate for 5-20 minutes at 10-20 rpm.
